(19) United States
(12) Patent Application Publication
(10) Pub. No.: US 2019/0321582 A1
(43) Pub. Date: Oct. 24, 2019

(54) Method and Device for Controlling Electrical Stimulation Therapeutic Apparatus

(71) Applicant: Zhonghua CI, Beijing (CN)

(72) Inventor: Zhonghua CI, Beijing (CN)

(21) Appl. No.: 15/958,920

(22) Filed: Apr. 20, 2018

(51) Int. Cl. A61M 21/02

(52) U.S. Cl. CPC A61M 21/02; A61M 2021/0027

(57) ABSTRACT

The present disclosure discloses a method and device for controlling an electrical stimulation therapeutic apparatus. The method includes: collecting voice information of a user, and acquiring a basic frequency of a user voice; determining a voice basic frequency interval of the user according to the basic frequency of the voice; acquiring music matched with the voice basic frequency interval; playing the music through a terminal connected to the electrical stimulation therapeutic apparatus and the electrical stimulation therapeutic apparatus; adjusting an electrical stimulation frequency of the electrical stimulation therapeutic apparatus according to the music. The present disclosure can make the electrical stimulation frequency of the electrical stimulation therapeutic apparatus change with the music frequency. Therefore, a current wearer will not develop tolerance and no adverse effects will be generated to the later therapeutic effect. The present disclosure can make patients condition sleeping, and condition aesthetic moods through music.

### TECHNICAL FIELD

The present disclosure relates to the field of intelligent health management, and particularly to a method and device for controlling an electrical stimulation therapeutic apparatus capable of relieving pressure and optimizing sleep.

### BACKGROUND

Currently, people's working pressure and living pressure are increasing, more and more people start to develop diseases such as depression, insomnia, and anxiety, and all of these diseases seriously affect people's normal work and life.

In order to treat patients suffering from these diseases so as to restore the normal work and life of the patients, an electrical stimulation therapeutic apparatus appears in the market. The electrical stimulation therapeutic apparatus is worn on ears of the patient. The electrical stimulation therapeutic apparatus has a therapeutic electrode, and when the electrical stimulation therapeutic apparatus works, the therapeutic electrode of the electrical stimulation therapeutic apparatus stimulates the patient with a stimulated current of a fixed frequency, for example, stimulates the patient with the stimulated current once every other second, or stimulates the patient with the stimulated current once every other two seconds, so as to achieve the object of treating the patient.

However, the inventor found that as the therapeutic electrode of the electrical stimulation therapeutic apparatus stimulates the patients with the stimulated current of the fixed frequency, the patients will develop tolerance after using the electrical stimulation therapeutic apparatus for a long period of time, which will produce adverse effects on the later therapeutic effect.

### SUMMARY

An object of the present disclosure is to provide a method and device for controlling an electrical stimulation therapeutic apparatus capable of changing a stimulation frequency with music so as to solve the problem existing in the prior art.

In order to achieve the above object, the present disclosure provides a method for controlling an electrical stimulation therapeutic apparatus, wherein the method includes:

collecting voice information of a user, and acquiring a basic frequency of a user voice;

determining a voice basic frequency interval of the user according to the basic frequency of the voice;

acquiring music matched with the voice basic frequency interval;

playing the music through a terminal connected to the electrical stimulation therapeutic apparatus and the electrical stimulation therapeutic apparatus;

adjusting an electrical stimulation frequency of the electrical stimulation therapeutic apparatus according to the music.

Furthermore, in the controlling method as described above, determining a voice basic frequency interval of the user according to the basic frequency of the voice includes:

acquiring an interval range of the voice basic frequency interval, the voice basic frequency interval being in one or more, and the voice basic frequency intervals having no overlapped region with each other;

matching the voice frequency with the voice basic frequency interval;

obtaining the voice basic frequency interval matched with the voice frequency.

Furthermore, in the controlling method as described above, acquiring music matched with the voice basic frequency interval includes:

acquiring music identification information of stored music, the music identification information including the voice basic frequency interval;

looking up for the music identification information corresponding to the voice basic frequency interval of the user;

acquiring music corresponding to the music identification information.

Furthermore, in the controlling method as described above, adjusting an electrical stimulation frequency of the electrical stimulation therapeutic apparatus according to the music includes:

acquiring the played music;

acquiring a music frequency of the music;

taking the music frequency as the basic input frequency, stimulating generation of a stimulated current of the electrical stimulation therapeutic apparatus according to the basic input frequency, the stimulated current changing the electrical stimulation frequency with changes of the basic input frequency.

Furthermore, in the controlling method as described above, acquiring a music frequency of the music includes:

acquiring a full music frequency of the music;

acquiring a music frequency interval section of 20 Hz˜20000 Hz in the full music frequency.

The present disclosure further provides a device for controlling an electrical stimulation therapeutic apparatus, including:

a voice frequency acquiring module, for collecting voice information of a user, and acquiring a basic frequency of a user voice;

a frequency interval matching module, for determining a voice basic frequency interval of the user according to the basic frequency of the voice;

a music matching module, for acquiring music matched with the voice basic frequency interval;

a music playing module, for playing the music through a terminal connected to the electrical stimulation therapeutic apparatus and the electrical stimulation therapeutic apparatus;

an electrical stimulation control module, for adjusting an electrical stimulation frequency of the electrical stimulation therapeutic apparatus according to the music.

Furthermore, in the controlling device as described above, the frequency interval matching module includes:

a first acquiring module, for acquiring an interval range of the voice basic frequency interval, the voice basic frequency interval being in one or more, and the voice basic frequency intervals having no overlapped region with each other;

a first matching module, for matching the voice frequency with the voice basic frequency interval;

a second acquiring module, for obtaining the voice basic frequency interval matched with the voice frequency.

Furthermore, in the controlling device as described above, the music matching module includes:

a third acquiring module, for acquiring music identification information of stored music, the music identification information including the voice basic frequency interval;

a first look-up module, for looking up for music identification information corresponding to the voice basic frequency interval of the user;

a fourth acquiring module, for acquiring music corresponding to the music identification information.

Furthermore, in the controlling device as described above, the electrical stimulation control module includes:

a fifth acquiring module, for acquiring played music;

a sixth acquiring module, for acquiring a music frequency of the music;

a first control module, for taking the music frequency as a basic input frequency, stimulating generation of a stimulated current of the electrical stimulation therapeutic apparatus according to the basic input frequency, the stimulated current changing an electrical stimulation frequency with changes of the basic input frequency.

Furthermore, in the controlling device as described above, the sixth acquiring module includes:

a music frequency acquiring sub-module, for acquiring a full music frequency of the music;

a frequency screening sub-module, for acquiring a music frequency interval section of 20 Hz˜20000 Hz in the full music frequency.

In the above technical solution, the present disclosure collects the voice information of the user, acquires the basic frequency of the user voice; determines the voice basic frequency interval of the user according to the basic frequency of the voice, and acquires music matched with the voice basic frequency interval; plays the music through the terminal connected to the electrical stimulation therapeutic apparatus and the electrical stimulation therapeutic apparatus; adjusts the electrical stimulation frequency of the electrical stimulation therapeutic apparatus according to the music, and is capable of determining a voice basic frequency interval of a current user according to this voice frequency. Since the music matched with different voice basic frequency intervals is already set in advance, after acquiring the voice basic frequency interval of the user, the music matched therewith can be acquired, and the music is played.

Thereafter, the electrical stimulation therapeutic apparatus acquires the music played by the terminal; acquires the music frequency of the music; adjusts the therapeutic electrical stimulation frequency of the electrical stimulation therapeutic apparatus according to the music frequency. Since the music frequency of the music is not fixed, but changes all the time, the stimulation frequency of the therapeutic electrode of the electrical stimulation therapeutic apparatus changes all the time, rather than is fixed all the time, therefore, a current wearer will not develop tolerance after using the electrical stimulation therapeutic apparatus for a long period of time, and no adverse effects will be generated to the later therapeutic effect. The present disclosure can make the patients use suitable types of music to condition sleeping, and condition aesthetic moods through music.

### BRIEF DESCRIPTION OF DRAWINGS

In order to more clearly illustrate technical solutions of examples of the present application or the prior art, figures which are needed for description of the examples will be introduced briefly below. Obviously, the accompanying drawings in the following description merely show some examples of the present disclosure. A person ordinarily skilled in the art still can obtain other accompanying drawings according to these accompanying drawings.

FIG. 1 is a flow chart of a method for controlling an electrical stimulation therapeutic apparatus of an example of the present disclosure;

FIG. 2 is an exemplary flow chart of step S2 of the method for controlling an electrical stimulation therapeutic apparatus shown in FIG. 1;

FIG. 3 is an exemplary flow chart of step S3 of the method for controlling an electrical stimulation therapeutic apparatus shown in FIG. 1;

FIG. 4 is an exemplary flow chart of step S5 of the method for controlling an electrical stimulation therapeutic apparatus shown in FIG. 1;

FIG. 5 is an exemplary flow chart of step S502 of the method for controlling an electrical stimulation therapeutic apparatus shown in FIG. 4;

FIG. 6 is a schematic view of a modular structure of a device for controlling an electrical stimulation therapeutic apparatus of an example of the present disclosure;

FIG. 7 is a schematic view of a modular structure of a five-note type acquiring module of an example of the device for controlling an electrical stimulation therapeutic apparatus shown in FIG. 5 of the present disclosure;

FIG. 8 is a schematic view of a modular structure of a music matching module of an example of the device for controlling an electrical stimulation therapeutic apparatus shown in FIG. 5 of the present disclosure;

FIG. 9 is a schematic view of a modular structure of an electrical stimulation control module of an example of the device for controlling an electrical stimulation therapeutic apparatus shown in FIG. 5 of the present disclosure;

FIG. 10 is a schematic view of a modular structure of a sixth acquiring module of an example of the device for controlling an electrical stimulation therapeutic apparatus shown in FIG. 9 of the present disclosure.

### DETAILED DESCRIPTION OF EMBODIMENTS

In order to enable a person skilled in the art to better understand the technical solutions of the present application, below the present disclosure will be further described in detail in conjunction with the accompanying drawings.

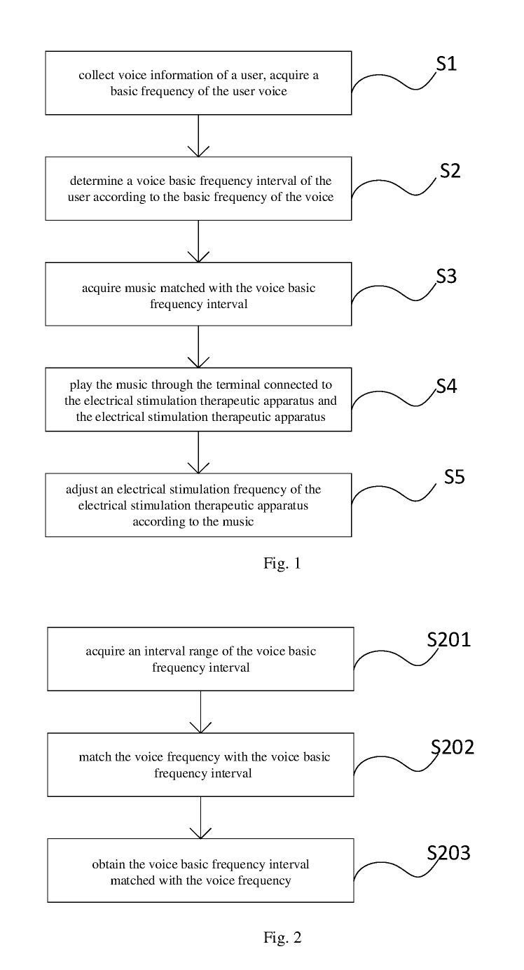

As shown in FIG. 1, the present disclosure provides a method for controlling an electrical stimulation therapeutic apparatus. The method includes:

S1: collecting voice information of a user, and acquiring a basic frequency of the user voice;

in this example, a terminal connected to the electrical stimulation therapeutic apparatus can be responsible for collecting the voice information of the user. The terminal can send or receive a signal through a wired or wireless network or other manners. Each terminal can be an electronic device including hardware, software, embedded logic components or a combination of two or more such kinds of components, and can carry out suitable functions implemented or supported by the user terminal. For example, the terminal can be a smart phone, a tablet computer, a portable E-mail device, an E-book, a hand-held game machine and/or a game controller, a notebook computer, a netbook, a hand-held electronic device, a smart wearable device, and so on.

In the above, the electrical stimulation therapeutic apparatus and the terminal can carry out data transmission therebetween through Bluetooth or other manners.

In the above, the user in the present text refers to a user wearing the electrical stimulation therapeutic apparatus on ears.

S2: determining a voice basic frequency interval of the user according to the basic frequency of the voice, and acquiring music matched with the voice basic frequency interval;

in this example, the voice basic frequency interval can be combined with the theory of 25 types of persons in the traditional Chinese medical science, with each voice basic frequency interval being corresponding to one type of person, and each type of person being corresponding to different physical symptoms.

In some examples, as shown in FIG. 2, the step S2 specifically includes the following steps:

S201: acquiring an interval range of the voice basic frequency interval, the voice basic frequency interval being in one or more, and the voice basic frequency intervals having no overlapped region with each other;

in the example of the present disclosure, a technician can set or divide each voice basic frequency interval in the terminal or server in advance, and store the same in the terminal or server.

Therefore, in the present step, when a corresponding table is located in the terminal, the terminal can directly locally acquire a correspondence relationship between the five-note type and the frequency interval; when the corresponding table is located in the server, the correspondence relationship between the five-note type and the frequency interval can be acquired from the server through the terminal or the electrical stimulation therapeutic apparatus.

S202: matching the voice frequency with the voice basic frequency interval, wherein in the matching, only the voice frequency and the voice basic frequency interval in the corresponding table need to be matched to obtain a corresponding voice basic frequency interval, and retrieve the corresponding voice basic frequency interval and take it as the voice basic frequency interval of the user.

S203: obtaining the voice basic frequency interval matched with the voice frequency.

S3: acquiring music matched with the voice basic frequency interval;

in the above, matching to obtain corresponding music can be carried out in a server side in network connection with the terminal or the electrical stimulation therapeutic apparatus, and also can be locally carried out in the terminal;

in some examples, as shown in FIG. 3, the step S3 specifically includes the following steps:

S301: acquiring music identification information of stored music, the music identification information including the voice basic frequency interval; if the music and the music identification information are stored on the terminal, a second correspondence relationship between the music and the five-note type can be directly acquired locally;

if the second correspondence relationship is not locally stored but stored in the server, the terminal or the electrical stimulation therapeutic apparatus can send to the server an acquiring request for acquiring the second correspondence relationship; when the server receives this acquiring request, the second correspondence relationship is acquired, and the second correspondence relationship is sent to the terminal; the terminal receives the second correspondence relationship sent by the server.

S302: looking up for the music identification information corresponding to the voice basic frequency interval of the user;

S303: acquiring music corresponding to the music identification information, wherein after the music identification information is found, the terminal needs to locally look up whether music corresponding to the music identification information exists, and when the music corresponding to the music identification information exists locally, the music corresponding to the music identification information is directly acquired locally; when the music corresponding to the music identification information does not exist locally, the music corresponding to the music identification information is downloaded from the server.

S4: playing the music through the terminal connected to the electrical stimulation therapeutic apparatus and the electrical stimulation therapeutic apparatus.

Means for realizing this method can include: 1. the electrical stimulation therapeutic apparatus directly plays the music that it downloads from the server; 2. the electrical stimulation therapeutic apparatus plays the music on the terminal through the Bluetooth connection with the terminal; 3. the music is directly played through the terminal.

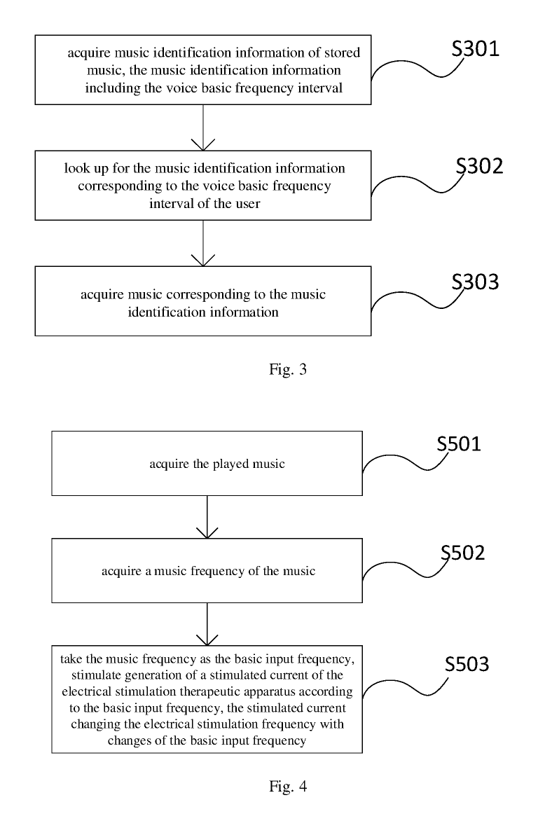

S5: adjusting an electrical stimulation frequency of the electrical stimulation therapeutic apparatus according to the music, wherein this method is used in electrical stimulation, and in some examples, as shown in FIG. 4, a specific flow of the step S5 includes the following steps:

S501: acquiring the played music;

in the above, the electrical stimulation therapeutic apparatus can acquire a signal of the music through detection, and also can directly analyze and process data of the music played by itself;

S502: acquiring a music frequency of the music;

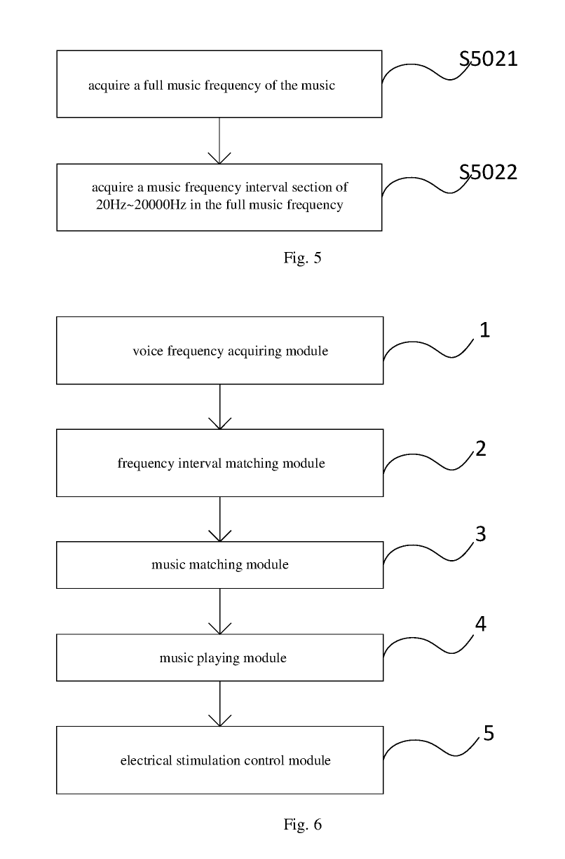

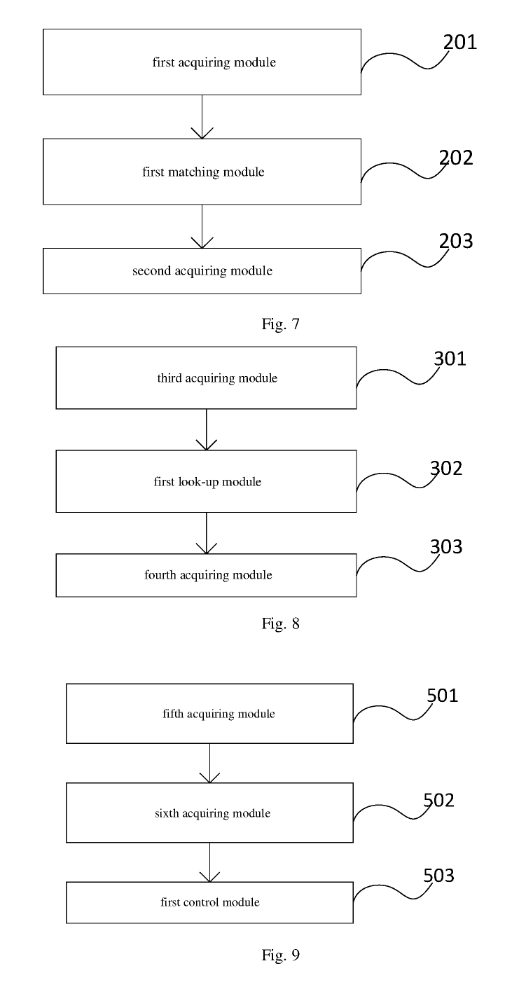

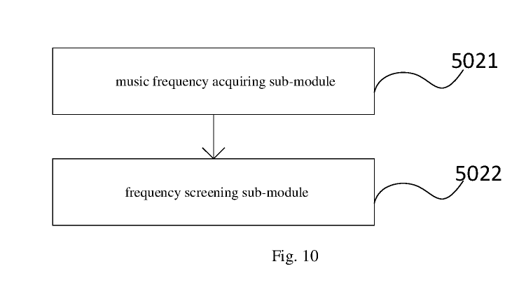

as shown in FIG. 5, in an example, the step S502 includes:

S5021: acquiring a full music frequency of the music;

S5022: acquiring a music frequency interval section of 20 Hz˜20000 Hz in the full music frequency, wherein since voices lower than 20 Hz or higher than 20000 Hz cannot be recognized by human ears, the music frequencies in these sections are deleted, so that the electrical stimulation frequency can be completely consistent with the music frequency, besides, preferably, murmur and noise portions in the music frequency are further deleted.

S503: taking the music frequency as the basic input frequency, stimulating generation of a stimulated current of the electrical stimulation therapeutic apparatus according to the basic input frequency, the stimulated current changing the electrical stimulation frequency with changes of the basic input frequency;

since the music frequency of the music is not fixed, but changes all the time, the electrical stimulation therapeutic apparatus stimulates the therapeutic electrode of the electrical stimulation therapeutic apparatus according to the music frequency to generate the stimulated current, so that the electrical stimulation frequency of the electrical stimulation therapeutic apparatus changes all the time, rather than is fixed all the time, therefore, the user will not develop tolerance after using the electrical stimulation therapeutic apparatus for a long period of time, and no adverse effects will be generated to the later therapeutic effect.

As shown in FIG. 6, the present disclosure further provides a device for controlling an electrical stimulation therapeutic apparatus, wherein the device includes:

a voice frequency acquiring module 1, for collecting voice information of a user, and acquiring a basic frequency of a user voice;

a frequency interval matching module 2, for determining a voice basic frequency interval of the user according to the basic frequency of the voice;

a music matching module 3, for acquiring music matched with the voice basic frequency interval;

a music playing module 4, for playing the music through a terminal connected to the electrical stimulation therapeutic apparatus and the electrical stimulation therapeutic apparatus;

an electrical stimulation control module 5, for adjusting an electrical stimulation frequency of the electrical stimulation therapeutic apparatus according to the music.

As shown in FIG. 6, in some examples, the frequency interval matching module 2 includes:

a first acquiring module 201, for acquiring an interval range of the voice basic frequency interval, the voice basic frequency interval being in one or more, and the voice basic frequency intervals having no overlapped region with each other;

a first matching module 202, for matching the voice frequency with the voice basic frequency interval;

a second acquiring module 203, for obtaining the voice basic frequency interval matched with the voice frequency.

In some examples, the music matching module 3 includes:

a third acquiring module 301, for acquiring music identification information of stored music, the music identification information including the voice basic frequency interval;

a first look-up module 302, for looking up for music identification information corresponding to the voice basic frequency interval of the user;

a fourth acquiring module 303, for acquiring music corresponding to the music identification information.

In some examples, the electrical stimulation control module 5 includes:

a fifth acquiring module 501, for acquiring played music;

a sixth acquiring module 502, for acquiring a music frequency of the music;

a first control module 503, for taking the music frequency as a basic input frequency, stimulating generation of a stimulated current of the electrical stimulation therapeutic apparatus according to the basic input frequency, the stimulated current changing an electrical stimulation frequency with changes of the basic input frequency.

In some examples, the sixth acquiring module 502 includes:

a music frequency acquiring sub-module 5021, for acquiring a full music frequency of the music;

a frequency screening sub-module 5022, for acquiring a music frequency interval section of 20 Hz˜20000 Hz in the full music frequency.

Specifically, for specific processes of realizing functions of each module in the present example, reference can be made to relevant description of the method example as described for FIG. 4, and unnecessary details will not be given herein.

The above merely describes some exemplary examples of the present invention in an illustrative manner. It goes without saying that a person ordinarily skilled in the art can modify the examples described through various different manners without departing from the spirit and scope of the present invention. Therefore, the above accompanying drawings and description are essentially illustrative, and should not be construed as limiting the scope protected by the claims of the present invention.

What is claimed is:

1. A method for controlling an electrical stimulation therapeutic apparatus, wherein the method comprises:
collecting voice information of a user, and acquiring a basic frequency of a user voice;
determining a voice basic frequency interval of the user according to the basic frequency of the voice;
acquiring music matched with the voice basic frequency interval;
playing the music through a terminal connected to the electrical stimulation therapeutic apparatus and the electrical stimulation therapeutic apparatus;
adjusting an electrical stimulation frequency of the electrical stimulation therapeutic apparatus according to the music.

2. The controlling method of claim 1, wherein determining a voice basic frequency interval of the user according to the basic frequency of the voice comprises:
acquiring an interval range of the voice basic frequency interval, the voice basic frequency interval being in one or more, and the voice basic frequency intervals having no overlapped region with each other;
matching the voice frequency with the voice basic frequency interval;
obtaining the voice basic frequency interval matched with the voice frequency.

3. The controlling method of claim 1, wherein acquiring music matched with the voice basic frequency interval comprises:
acquiring music identification information of music stored in advance, the music identification information comprising the voice basic frequency interval;
looking up for the music identification information corresponding to the voice basic frequency interval of the user;
acquiring music corresponding to the music identification information.

4. The controlling method of claim 1, wherein adjusting an electrical stimulation frequency of the electrical stimulation therapeutic apparatus according to the music comprises:
acquiring the played music;
acquiring a music frequency of the music;
taking the music frequency as the basic input frequency, stimulating generation of a stimulated current of the electrical stimulation therapeutic apparatus according to the basic input frequency, the stimulated current changing the electrical stimulation frequency with changes of the basic input frequency.

5. The controlling method of claim 4, wherein acquiring a music frequency of the music comprises:
acquiring a full music frequency of the music;
acquiring a music frequency interval section of 20 Hz˜20000 Hz in the full music frequency.

6. A device for controlling an electrical stimulation therapeutic apparatus, comprising:
a voice frequency acquiring module, for collecting voice information of a user, and acquiring a basic frequency of a user voice;
a frequency interval matching module, for determining a voice basic frequency interval of the user according to the basic frequency of the voice;
a music matching module, for acquiring music matched with the voice basic frequency interval;
a music playing module, for playing the music through a terminal connected to the electrical stimulation therapeutic apparatus and the electrical stimulation therapeutic apparatus;
an electrical stimulation control module, for adjusting an electrical stimulation frequency of the electrical stimulation therapeutic apparatus according to the music.

7. The controlling device of claim 6, wherein the frequency interval matching module comprises:
a first acquiring module, for acquiring an interval range of the voice basic frequency interval, the voice basic frequency interval being in one or more, and the voice basic frequency intervals having no overlapped region with each other;
a first matching module, for matching the voice frequency with the voice basic frequency interval;
a second acquiring module, for obtaining the voice basic frequency interval matched with the voice frequency.

8. The controlling device of claim 6, wherein the music matching module comprises:
a third acquiring module, for acquiring music identification information of stored music, the music identification information comprising the voice basic frequency interval;
a first look-up module, for looking up for music identification information corresponding to the voice basic frequency interval of the user;
a fourth acquiring module, for acquiring music corresponding to the music identification information.

9. The controlling device of claim 6, wherein the electrical stimulation control module comprises:
a fifth acquiring module, for acquiring played music;
a sixth acquiring module, for acquiring a music frequency of the music;
a first control module, for taking the music frequency as a basic input frequency, stimulating generation of a stimulated current of the electrical stimulation therapeutic apparatus according to the basic input frequency, the stimulated current changing an electrical stimulation frequency with changes of the basic input frequency.

10. The controlling device of claim 9, wherein the sixth acquiring module comprises:
a music frequency acquiring sub-module, for acquiring a full music frequency of the music;
a frequency screening sub-module, for acquiring a music frequency interval section of 20 Hz˜20000 Hz in the full music frequency.
